(19) United States
(12) Patent Application Publication
(10) Pub. No.: US 2017/0138940 A1
(43) Pub. Date: May 18, 2017

(54) METHOD FOR ISOLATING TRACE COMPONENTS FROM A BIOLOGICAL LIQUID SAMPLE

(71) Applicant: MagnaMedics GmbH, Aachen (DE)

(72) Inventors: Sven Goethel, Troisdorf (DE); Viorel Rusu, Eygelshoven (NL); Erik Ruijters, Maastricht (NL); Tim Welten, Geleen (NL)

(21) Appl. No.: 15/335,890

(22) Filed: Oct. 27, 2016

Related U.S. Application Data
(63) Continuation of application No. PCT/DE2015/000199, filed on Apr. 27, 2015.

(30) Foreign Application Priority Data
Apr. 28, 2014 (DE) 10 2014 005 993.6

(51) Int. Cl. G01N 33/543; G01N 33/552

(52) U.S. Cl. CPC G01N 33/54326; G01N 33/552

(57) ABSTRACT

A method for isolating trace components selected from various groups and substances as set forth in the disclosure includes a) lysis of the cells and/or b) depletion of proteins by binding on mobile, magnetic silica gel beads and/or c) removal of peptides, amino acids, sugars, lipids, phospholipids and inorganic salts by binding on mobile, magnetic silica gel beads. The binding on the mobile, magnetic silica gel beads under b) and/or c) takes place non-selectively, selectively and/or reversibly, and the isolation of the trace components takes place timely offset after steps b) and/or c) with the aid of magnetic separation by binding on the surface of the mobile, magnetic silica gel beads, which differ from the mobile, magnetic silica gel beads under b) and/or c) in terms of different functional groups.

### REFERENCE TO RELATED APPLICATIONS

This application is a continuation of International Application number PCT/DE2015/000199 filed on Apr. 27, 2015, which claims priority to German Application number 10 2014 005 993.6 filed on Apr. 28, 2014.

### FIELD

The disclosure relates to a method and a kit for sample preparation for the isolation of trace components in chemical analysis and diagnostics from a liquid biological sample by depletion of high-molecular-weight compounds (1) and removal of low-molecular-weight compounds (2).

### BACKGROUND

Liquid biological materials such as whole blood, serum, saliva, urine or plasma and other clinically relevant liquids are typically tested by means of immunoassays, especially ELISA assays, for the quantitative content of low-molecular-weight, organic compounds in order to record a disease's progression, to diagnose a disease in the first place or to determine the medicament level. The trace components which are to be analyzed can be naturally occurring compounds (e.g., vitamins or metabolites), but can also be exogenously administered substances such as medicaments or drugs.

In recent years, LC-MS (or LC-MS/MS) has started to establish itself as an alternative to immunoassays. Typically, this involves removing the clinical sample by means of precipitation and subsequent centrifugation of plasma proteins and then separating and quantifying the low-molecular-weight compounds in a reversed-phase LC (area of the LC graph) and analyzing the molar mass by mass spectrometry (MS).

Although highly sensitive and accurate measured results can be achieved using mass spectrometry, there are numerous substances which have an adverse effect on the measured result. Since said substances may originate from a matrix such as, for example, whole blood, serum, but also plant extracts or foodstuffs, reference is also made to so-called adverse matrix effects. In the case of the substances to be mentioned, a distinction must be made between high-molecular-weight compounds such as, for example, proteins and low-molecular-weight compounds such as, for example, phospholipids, since a depletion or removal of both substance classes is not possible with a single physical/chemical method.

It is known that, depending on the nature of the sample and the test goal, the qualitative and quantitative analyse of a target compound or of a trace component such as, for example, a biomarker target from a biological sample are carried out on different instrumental platforms and for different end uses such as clinical inspections, doping tests, forensic/toxicological reports or food inspections. In this connection, pretreatments of the sample are necessary, regardless of the method of testing to be carried out, such as, for example, for immunoassays (ELISA) or mass spectroscopy, in order to minimize interfering components such as, for example, matrix effects.

The analytical process from sample collection up to the final result is essentially divided into five steps:

1. Sample collection
2. Sample preparation
3. Sample fractionation
4. Analyte detection
5. Data evaluation

However, biological samples, as present for example in forensics and in the pharmaceutical industry as raw materials and final products, are multicomponent systems having a highly complex composition which frequently contains fatty acids, proteins, salts, lipids, acids, bases and organic compounds as constituents. Some of these compounds can, as constituents of samples, be considered to be “quasi” matrix, i.e., they have similar properties to the trace components.

Moreover, the trace components in pending analyses are most of the time present in a low concentration in the sample. Therefore, the aim is to make it possible, depending on the origin of the samples and the analytical goals, to separate the biological samples, but also drug analyses, into various constituents and to isolate or enrich specific components such as, for example, trace components.

### BRIEF DESCRIPTION OF THE SEVERAL VIEWS OF THE DRAWINGS

FIG. 1 shows various schematics illustrating example methodologies for simultaneously or separately isolating trace components in one or more cavities utilizing a kit of the present disclosure.

FIG. 2a shows an example graph illustrating a sample preparation according to protocol 2 of Example 8 of the present disclosure.

FIG. 2b shows an example graph illustrating a sample preparation according to protocol 1 of Example 8 of the present disclosure.

FIG. 3 shows an example calibration curve for an example antidepressant.

FIG. 4 shows an example chromatogram of substances from a urea-containing solution that are determined using the method of the present disclosure.

### DETAILED DESCRIPTION

The disclosure provides a method for sample preparation having positive selection of trace components with negative selection of abundant compounds of a sample using magnetic beads for sample preparations.

According to the disclosure, a method and a kit for sample preparation comprises the following features:
a) lysis of the cells and/or
b) depletion of compounds (1) from a liquid biological sample by binding on a solid-phase support and/or
c) removal of compounds (2) by binding on a mobile solid-phase support,

it being possible for the binding on the mobile solid-phase supports under b) to c) to take place selectively and/or reversibly.

The present disclosure provides a method for isolating trace components in biological samples, it being possible for a depletion or removal of various types of compounds (1) and (2) to take place. Compounds (1) can, for example, encompass abundant proteins. Furthermore, applications which are effected in the area of sample preparation in kit-based format are also possible. In particular, the present disclosure makes it possible to regulate a selective isolation of trace components in the presence of a protein-rich matrix (for example of hormones, vitamins, especially vitamin D, immunosupressants, mycotoxins, etc.) using controlled depletion of proteins in the presence of silica gel beads, especially of silica gel beads having magnetic cores.

The disclosure is directed to the field of sample preparation for UHPLC, HPLC, LC/MS, LC/MS/MS, capillary LC, capillary electrophoresis, immunoassays incl. ELISA and other assays which are oriented toward the sample preparation methods for the detection of trace components and/or low-molecular-weight compounds for analyses in complex clinical and nonclinical samples. Clinical samples in the context of the disclosure are directed to homogenized tissue, whole blood, cell culture, plasma, serum, urine, saliva, tears, spinal fluid, tissue fluid, amniotic fluid, follicular fluid and hemolyzed blood. Nonclinical samples are directed to foodstuffs, beverages, water samples, liquid environmental samples and fermentation media.

As for an appropriate therapy an appropriate diagnosis of the diseases is required, the aim being to carry out a rapid diagnosis in order to be able to make clinical decisions with good prospects. Thus, in addition to the possibility of processing a biological sample with respect to the trace components present therein, the disclosure also envisages providing a method which makes it possible to isolate the trace components and to subsequently analyze them.

In accordance with the method according to the disclosure, compounds (1) and compounds (2) can, surprisingly, be depleted or removed from the biological liquids in a simple and rapid manner without the compositions or the quantities of the trace components from the sample being altered. This is true at least for the circumstance of the trace components not being concentrated in a final cleanup step.

Compounds (1) and (2) which can be depleted and/or removed in the context of the present disclosure can, for example, be serum albumins, immunoglobulins, fibrinogen, regulator proteins.

Furthermore, the method according to the disclosure makes it possible to isolate or remove sugars, lipids, phospholipids and inorganic salts too as compounds (2). After analysis of the relevant target compounds such as, for example, of trace components in the form of medicaments or biomarker targets, the method according to the disclosure makes it possible to make clinical decisions which can be translated into a therapeutic success. Such clinical decisions then encompass further treatments, if necessary, using medicaments for the therapy of diseases.

In the method according to the disclosure, tissue samples (biopsy) or body fluid (blood, plasma) can be removed from the patient to be examined, it being possible for the diagnosis to take place in vitro/ex vivo, i.e., outside the human or animal body. Owing to the determination of the trace components according to the disclosure, great significance is attained for existing quantities or their increase/decrease.

In the context of the method according to the disclosure, depletion of a compound means a reduction of high-molecular-weight compounds (1) from a biological sample. This can be effected by binding, such as adsorption for example, to a mobile solid-phase support, which can, for example, consist of magnetic silica gel, preferably of mesoporous silica gel, but also polystyrene, melamine, polymethacrylate, polyamide, polyvinyl chloride, polyethylene, polypropylene, polyester, polycarbonate, polyacrylamide, agarose, chitin, dextran, rubber and polyvinyl alcohol.

According to the disclosure, removal is to be understood to mean the binding of compounds (2) to mobile solid-phase supports. In this case, the mobile solid-phase supports can have various designs like those which are used for the depletion of compounds (I), i.e., they can all contain the same dimensions, surfaces and core constituents or else have different designs among each other. The design of the mobile solid-phase supports is guided by the type, size and quantity of the compounds (1) and (2) present in the sample.

The further purification of biological samples besides compounds (1) and (2) includes the isolation of trace components, which can be effected by selective and reversible binding to mobile solid-phase supports, for example in the form of silica gel beads which can have a surface-modified structure. For this purpose, it is possible, though not necessary, to use silica gel beads as mobile solid-phase supports. The isolation of the trace components, for example in the residue, can also be effected after carrying out the removal or depletion of compounds (1) and (2). The silica gel beads can include individual or multiple types of surface structures, the binding of the trace components to the mobile solid-phase supports taking place by means of ionic, hydrophobic, π-π interactions, and also metal-ion interactions, and it being possible to establish a reversible bond.

In a particular design, the mobile solid-phase supports comprise magnetic beads, for example in the core, and comprise on the surface a mixture of various molecular chains which have two main functions: firstly, acting as mobile solid-phase support for the depleted or removed compounds (1) and (2) and, secondly, being available as mobile solid-phase support for the isolation of trace components, it being possible for the particular binding of the mobile solid-phase supports in relation to compounds (1) and (2) and the trace components to be dependent on different and specific interactions.

To carry out the method according to the disclosure, compounds (1) and/or (2) are bound to a mobile solid-phase support such as, for example, an adsorbent composed of silica gel beads having a magnetic core and surface structures which can have functional groups such as NH₂ and COOH. In this case, the silica gel beads have pores, the size of which is within the range from 2 to 50 nm, preferably 5 to 30 nm, and the inner surface of which comprises a range from 0.1 to 400 m²/g, preferably 10 to 200 m²/g.

In general, the beads have a diameter within the range from 20 nm to 500 μm, preferably from 50 nm to 100 μm, particularly preferably from 100 nm to 10 μm.

In the context of the present disclosure, protic and aprotic polar organic solvents are used for binding and/or elution. These generally have a dipole moment within the range from 1.6 to 4.0 Debye, preferably from 1.69 to 3.96 Debye.

Suitable polar organic solvents include the following, these being examples and not a conclusive list: acetonitrile, ethanol, methanol, propanol, isopropanol, n-propanol, isobutanol and n-butanol, and also acetone, dimethyl sulfoxide and polyethylene glycol (PEG).

The polar, organic solvents can be used individually or as a mixture. The mixtures are, for example, set such that alcohols such as ethanol and isopropanol in various proportions by weight or acetonitrile together with an alcohol such as, for example, isopropanol or ethanol or combinations of alcohols such as ethanol and isopropanol having various proportions by weight with, for example, acetonitrile are combined.

To further purify or to further concentrate the trace components, they can, in a further step, be gathered in nonpolar, aprotic solvents, such as hexane for example, by liquid-liquid extraction and, after subsequent evaporation of the solvent, the residue can be gathered again in a suitable polar solvent such as, for example, methanol or acetonitrile.

The following substances or substance classes are to be counted as trace components or biomarker targets in the context of the disclosure, these being examples and not a conclusive list:

cardiovascular agents, such as amiodarone, digoxin, digitoxin, flecainide, lidocaine, N-acetylprocainamide, procainamide, propranolol,

immunosuppressants, cyclosporine A, tacrolimus, sirolimus, everolimus, antiarrhythmic agents, such as acebutolol, ajmaline, desethylamiodarone, aprodine, atenolol, bisopropol, quinidine, diltiazem, disopyramide, dronedarone, flecainide, flunarizine, gallopamil, sotalol, tocainide, verapamil, norverapamil,

for the central nervous system, such as carbamazepine, carbamazepine-10,11-epoxide, carbamazepine-diol, 10-OH carbamazepine, ethosuximide, felbamate, phenobarbital, free carbamazepine, free phenytoin, free primodone, free valproic acid, gabapetin, lacosamide, lamotrigine, levetiracetam, oxcarbazepine, phenylethylmalonamide (PEMA), phenobarbital, pregabalin, rufinamide, stiripentol, sulthiame, tiagabine, topiramate, theophylline, vigabatrin, zonisamide, phenytoin, primidone, valproic acid,

antibiotics, such as vancomycin, amikacin, chloramphenicol, gentamicin, kanamycin, metilmicin, streptomycin,

antimycotics, such as itraconazole, fluconazole, posaconazole, voriconazole, 5-flucytosine, hydroxyitraconazole,

antidepressants, such as O-desmethylvenlafaxine, mirtazapine, venlafaxine, citalopram, paroxetine, desmethylfluoxetine, fluoxetine, fluvoxamine, duloxetine, sertraline, N-desmethylsertraline, methylphenidate/ritalin, reboxetine, trazodone, mianserin, atomoxetine,

neuroleptics, such as N-desmethylolanzapine, olanzapine, 9-OH risperidone, risperidone, haloperidol, quetiapine, clozapine, N-desmethylclozapine, aripiprazole, amisulpride, pipamperone, promethazine, levomepromazine, perazine, chlorprothixene, ziprasidone,

tricyclic antidepressants, such as amitriptyline, chlorpromazine, cyclobenzaprine, clozapine, norclozapine, doxepin, perphenazine, imipramine, clomipramine, trimipramine,

benzodiazepines, such as aminoclonazepam, aminoflunitrazepam, hydroxyalprazolam, hydroxytriazolam, brotizolam, clobazam, clonazepam, chlordiazepoxide, clorazepate, clotiazepam, cloxazolam, diazepam, ketazolam, midazolam, nordiazepam, oxazepam, prazepam, temazepam, tetrazepam, zolpidem, alprazolam, bromazepam, flunitrazepam, flurazepam, lorazepam, lormetazepam, nitrazepam, triazolam,

drugs, such as amphetamine, barbiturate, cannabinoids, cocaine, lysergic acid diethylamide, morphine, opiates, propoxyphene, fentanyl, sufentanil, ephedrine, ethyl glucuronide,

hormones, female estrogen, male estrogen, male testosterone, female testosterone, female progesterone, male progesterone, steroids, such as cortisol, ACTH, aldosterone, dehydroepiandrostenedione (DHEA), dehydroepiandrostenedione sulfate (DHEAS), androstenedione, 17-OH progesterone, 11-deoxycortisol, corticosterone,

fat-soluble vitamins, such as vitamin A, vitamin E, vitamin K, 25-OH vitamin D3, 25-OH vitamin D2,

B vitamins, such as vitamin B1, vitamin B6,

metabolic products, such as methylmalonic acid, folic acid, arachidonic acid, prostaglandin E2, homocysteine,

catecholamines, such as noradrenaline (norepinephrine), adrenaline (epinephrine), dopamine, serotonin

Additionally tumor markers, psychopharmaceuticals, cardiovascular medicaments, insecticides, pesticides, fungicides, antibodies, vasopressin, oxytocin, somatostatin, enkephalin, beta-endorphin, thyroxine, triiodothyronine, thyroglobulin, calcitonin, catecholamine, parathormone, aldosterone, renin, angiotensin, cortisone, deoxycorticosterone, androsterone, pregnenolone, estrone, estradiol, estriol, FSH, gonadotropin, tacrolimus, everolimus, MPA, insulin, anti-insulin antibodies, glucagon, gastrin, secretin, AMP, GMP, MMP 20, ZAP 1, ZAP 2, ZAP 3, prostaglandins, thromboxane, erythropoietin, histamine, primidone, acetazolamide, sultiame, glutethimide, pentobarbital, secobarbital, bupivacaine, mepivacaine, quinidine, amikacin, gentamicin, tobramycin, cefalexin, sulfamethoxazole, methotrexate, cyclosporine, methylprednisolone, salicylic acid, acetaminophen, indomethacin, allopurinol, carotene, vitamin C, vitamin D, 25-OH vitamin D, angiotensin I and II, hemopexin, transferrin, cortisol, insulin, fibrinogen, antithrombin, plasminogen, antiplasmin, etc.

The mobile solid-phase supports can comprise silica gel beads which can be designed with at least one magnetic core. As a result of coating the iron oxide-doped core, the beads, to which silica gel can be applied for example, comprise functional groups such as, for example, —OH, —COOH, —NH₂, R—SO₂—OH, —NH₂; —RNH, —R₂N, alkyl such as CH₃, —C₂H₅, —C₄H₉, —C₈H₁₇, —C₉H₁₉, —C₁₀H₂₁, -C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₆H₅, —ZrO₂, TiO₂, C₆H₉NO₆, phenylhexyl, biphenyl, hydroxyapatite, boronic acid.

The abovementioned functional groups achieve interactions which can ensure an optionally reversible binding of compounds (1) and (2) and/or trace components on the surface of the mobile solid-phase supports. These include ionic interactions, hydrogen bonds, hydrophobic interactions, π-π interactions and also metal-ion interactions. The abovementioned interactions allow an induced adhesion of compounds (1) and (2) and/or of the trace components on the surface of the mobile solid-phase supports, especially when using polar solvents.

Coating of iron-oxide-containing beads with silica gel is known per se (J. Colloid Interface Sci. 1968, 26, 62 to 69; Langmuir 2005, 21, 10763 to 10769; J. Colloid Interface Sci 2005, 283, 392 to 396).

Coating of iron-oxide-containing beads with silica gel for the method according to the disclosure can, for example, be carried out as follows:

A suspension of iron-oxide-containing beads in an alcohol (e.g., isopropanol) is mixed with tetraethyl orthosilicate (TEOS) with vigorous stirring in the presence of ammonia in order to achieve coating. The thickness of the coating can be controlled by the quantity of the tetraethyl orthosilicate added.

The coated iron-oxide-containing beads are washed with an alcohol (e.g., methanol) and stored in water.

For the method according to the disclosure, particular preference is given to silica gel beads as mobile solid-phase supports, consisting of a mesoporous layer which has been applied to the magnetic core and has a layer thickness within the range from 10 to 100 nm.

Magnetic cores for the silica gel bead mobile solid-phase supports according to the disclosure can comprise iron oxide (Fe₂O₃ or Fe₃O₄) beads, which are known per se, and have a surrounding layer composed of, for example, silicon dioxide, but also polystyrene, melamine, polymethacrylate, polyamide, polyvinyl chloride, polyethylene, polypropylene, polyester, polycarbonate, polyacrylamide, agarose, chitin, dextran, rubber and polyvinyl alcohol.

For the method according to the disclosure, preference is given to using iron oxide beads having a mesoporous silica gel coating, as arises, for example, in the presence of polyethylene glycol as porogenic agent.

Particular preference is given to silica gel beads consisting of a mesoporous layer which has been applied to the magnetic core and has a layer thickness within the range from 10 to 100 nm, the magnetic cores containing maghemite and/or magnetite within the range from 30 to 95% by weight and having a mean diameter within the range from 20 nm to 500 μm, preferably from 200 nm to 10 μm, particularly preferably from 300 nm to 5 μm.

The method according to the disclosure can be carried out such that a selective and/or reversible binding of compounds (1) and (2) to the mobile solid-phase supports takes place, i.e., a binding of compounds (1) and (2) to a mobile solid-phase support takes place for the depletion of compounds (1) and removal of compounds (2) with simultaneous isolation of the trace components, with the trace components remaining in solution. The advantage of this is that, depending on the task, it is possible to perform a rapid and selective isolation of trace components by parallel binding of compounds (1) and (2) to the mobile solid-phase supports without obtaining multistep fractions. The trace components obtained can then be subjected to a subsequent analysis.

However, it is also possible for compounds (1) and (2) to first be bound to mobile solid-phase supports and for the trace components remaining in the solution to then be bound to mobile solid-phase supports. The mobile solid-phase supports can differ according to the size and nature of the functional groups, and so only particular compounds (1) and (2) or trace components bind to the differently designed mobile solid-phase supports. The method steps for the binding of the individual sample constituents can be carried out simultaneously, i.e., in parallel, or successively. After an optional elution step, the trace components thus obtained can then be subjected to a subsequent analysis.

In addition, a further embodiment of the subject matter of the disclosure envisages that only compounds (2) are removed first of all, compounds (1) are cleared from the sample in a subsequent depletion, and the trace components remaining in the solution are then subjected to an analysis. Alternatively, the trace components can also be bound to mobile solid-phase supports having a particular surface design, for example through the appropriate selection of the functional groups. After an optional elution step, the bound trace components can then be subjected to a subsequent analysis.

It is also envisaged to first generate a binding of the trace components to suitable mobile solid-phase supports and to then perform a depletion of compounds (2) and removal of compounds (1) from the sample. After an elution step, the trace components can then be subjected to an analysis.

In this respect, the depletion of compounds (1) and removal of compounds (2) can be carried out with the isolation of the trace components in the sample after a delay or at the same time, it being possible to vary the design of the method steps depending on the nature of the task.

In a preferred embodiment of the method according to the disclosure, a buffer solution of low ionic strength is used in the lysis of the cells in a first method step a), the proportion by volume of the biological sample in relation to the buffer solution being from 1:2 to 1:10 and the buffer solution having a molar salt content of from 0 to 0.5 mol/l. In a further method step b), 0.5 to 2.5 parts by volume of a buffer solution of low ionic strength is added to one part by volume of the biological sample, which buffer solution contains a divalent salt having a molar concentration of up to one mol/l, the divalent salt being especially zinc sulfate.

The removal of the silica gel beads containing at least one magnetic core is achieved with the aid of a magnetic separator.

The method according to the disclosure can, for example, be carried out as in the following steps:
(i) providing a liquid biological sample containing one or more trace components, and also compounds (1) and/or compounds (2),
(ii) contacting the liquid biological sample with silica gel beads containing a magnetic core,
(iii) mixing in organic solvents in a predefined ratio
(iv) swirling and incubating the mixture, with compounds (1) and/or compounds (2) being adsorbed on the surface of the beads,
(v) removing the magnetic beads by application of a magnetic field, and
(vi) taking off the supernatant containing one or more trace components in solution or suspension. Alternatively, extraction of one or more compounds (1) and (2) and/or of trace components from the sample with subsequent elution and analysis.
(vii) Optionally drying the supernatant or the elution solution or liquid by evaporation of the organic solvent, mixing at elevated temperature (50-85° C.) or in a gas stream and
(viii) analyzing the one or more compounds (1) and (2) and/or trace components using gel electrophoresis, UHPLC, HPLC, UPLC, LC/MS, LC-MS/MS, capillary LC, capillary electrophoresis, UV/Vis, immunoassay detection and/or flow cytometry.

Optionally, a few other chromatographic techniques can also be carried out within the analytical chain prior to the mass spectroscopy.

The present disclosure also provides analyses of samples containing a low content of trace elements, with the compounds from a sample being adsorbed to silica gel beads containing a magnetic core as a result of a change in the dielectric constant with the aid of polar, organic solvents having a dipole moment within the range from 1.6 to 4.0 Debye and the magnetic silica gel beads containing the adsorbed compounds being removed in a magnetic field.

Binding or attachment on the surface of the solid-phase support can be achieved by various mechanisms. For instance, it is possible for the compounds to be bound on the surface by various interactions, and so, in the course of a subsequent sample processing, the trace components bound by adsorption can optionally be easily released from the surface. The interactions which ensure optionally a reversible binding of the compounds on the surface include ionic interactions, hydrogen bonds, hydrophobic interactions, π-π interactions and metal-ion interactions. The abovementioned interactions allow an induced adhesion of the compounds on the surface of the adsorbent, especially when using polar solvents. As a result of a specific change in the pH, the salt concentration, the polarity of the solvent, the induced adhesion is made irreversible and the trace component becomes eluted from the solid-phase support. Besides a purification of the sample, a concentration of the trace component(s) can thus also be achieved.

The method according to the disclosure also includes the combination of the selective isolation of the trace components and of the depletion and/or removal of compounds (1) and (2). The combination is usually, but not exclusively, achieved in a two-step method. In this case, compounds (1) and (2) are bound to the solid-phase support by selective and nonselective bonds and thus cleared from the reaction mixture. In a further step, the trace components are selectively and reversibly bound to a further solid-phase support and optionally eluted from the solid-phase support by varying the solvent (pH, salt concentration, polarity of the solvent).

In this connection, the steps can take place simultaneously in one reaction chamber, but also at different times and in two or more cavities. Especially in the chemical analysis of trace components in addition to proteins and interfering low-molecular-weight compounds as main component, the present disclosure advantageously affords a simple execution of the chemical analysis with minimal errors, which can be caused especially by the complexity of the system and the matrix effects. In one embodiment of the disclosure, the method according to the disclosure can be carried out in the context of an in vitro diagnosis by means of parallel or simultaneous determinations of the markers according to the disclosure, with the determinations being carried out on at least one patient sample. In a further embodiment of the disclosure, the method according to the disclosure can be carried out with the aid of 2D electrophoresis, with isoelectric focusing being carried out in the first dimension and gel electrophoresis being carried out in the second dimension.

In a further embodiment, the method according to the disclosure and its determinations can be carried out by means of a rapid test (e.g., lateral flow test), either in single-parameter determination or multiparameter determination.

Lastly, the present disclosure includes an application package or kit for sample preparation for clinical samples, the constituents used in the kit comprising lysis buffers, organic solvents and magnetic solid-phase supports. In this case, the constituents present in the kit are to be used similarly to the constituents described in the method according to the disclosure in a concentration range between 1 to 15 percent by volume. Different clinical applications for the kit are possible as a result, it being possible to analyze various sample types especially through the use of variable concentration ranges of the existing kit constituents.

A few typical uses of the method and kit according to the disclosure for sample preparation are listed below by way of example.

### Example 1

### Sample Preparation for the Isolation and Analysis of Cortisol in Human Whole Blood:

### Materials:

Lysed whole blood containing cortisol
Cortisol solution: 250 μg/ml in sterile water
Magnetic silica gel beads (magnetic bead mix (50 mg/ml)) in aqueous zinc sulfate solution (0.4 M)
Organic solvent mix, 100% acetonitrile
Microtiter plates, UV/Vis-compatible
Microtiter plate reader
1. In microtiter plate format, aqueous cortisol solution is added in different concentrations to the whole blood, mixed, and incubated for 1 min for the purpose of cell lysis.
2. Zinc-sulfate-containing bead mix is added to the reaction preparation, mixed, and incubated for one minute.
3. Addition of acetonitrile to the reaction preparation precipitates the proteins at the magnetic bead mix.
4. The added or generated solids are removed in their entirety by a suitable magnetic separator and the supernatant solution is taken off for further analysis.
5. The supernatant is measured in the microtiter plate reader at 240 nm and the optical density (OD₂₄₀) is recorded.

One finding is that lysed whole blood has, at 240 nm, a background absorption independent of the cortisol added. Therefore, the absorption values of cortisol-containing samples must be subtracted (corrected) by the blank value.

### A Pipetting Scheme is Listed Below:

| | Whole | | Cortisol | | Acetonitrile | |
|---|---|---|---|---|---|---|
| Pipetting | blood | Water | sol. | Bead sol. | sol. | |
| scheme: | [μl] | [μl] | [μl] | [μl] | [μl] | Total |
| 1 | 50 | 250 | 0 | 50 | 700 | 1050 |
| 2 | 50 | 250 | 0 | 50 | 700 | 1050 |
| 3 | 50 | 210 | 40 | 50 | 700 | 1050 |
| 4 | 50 | 210 | 40 | 50 | 700 | 1050 |
| 5 | 50 | 170 | 80 | 50 | 700 | 1050 |
| 6 | 50 | 170 | 80 | 50 | 700 | 1050 |
| 7 | 50 | 130 | 120 | 50 | 700 | 1050 |
| 8 | 50 | 130 | 120 | 50 | 700 | 1050 |
| 9 | 50 | 90 | 160 | 50 | 700 | 1050 |
| 10 | 50 | 90 | 160 | 50 | 700 | 1050 |
| 11 | 50 | 50 | 200 | 50 | 700 | 1050 |
| 12 | 50 | 50 | 200 | 50 | 700 | 1050 |

### Readout:

| | | Cortisol in | Mean blank | | Rel. abs. | Deviation |
|---|---|---|---|---|---|---|
| | | assay | value | Corr. | coeff. | alpha in |
| | OD₂₄₀ | [μg] | [OD₂₄₀] | OD₂₄₀ | [alpha] | [%] |
| 1 | 0.3449 | 0 | 0.3619 | 0.0171 | n.a. | |
| 2 | 0.3790 | 0 | 0.3619 | 0.0171 | n.a. | |
| 3 | 0.5713 | 10 | 0.3620 | 0.2094 | 0.1099 | 96.9 |
| 4 | 0.5972 | 10 | 0.3620 | 0.2353 | 0.1235 | 108.9 |
| 5 | 0.8047 | 20 | 0.3620 | 0.4428 | 0.1162 | 102.4 |
| 6 | 0.7806 | 20 | 0.3620 | 0.4187 | 0.11 | 96.9 |
| 7 | 0.9878 | 30 | 0.3620 | 0.6259 | 0.1095 | 96.5 |
| 8 | 1.0328 | 30 | 0.3620 | 0.6709 | 0.1174 | 103.5 |
| 9 | 1.2283 | 40 | 0.3620 | 0.8664 | 0.1137 | 100.3 |
| 10 | 1.2395 | 40 | 0.3620 | 0.8776 | 0.1151 | 101.6 |
| 11 | 1.3627 | 50 | 0.3620 | 1.0008 | 0.1050 | 92.6 |
| 12 | 1.4440 | 50 | 0.3620 | 1.0821 | 0.1136 | 100.2 |
| | | | | Mean | 0.1134 | |

The recovery rate of cortisol added additively in Example 1 is 92%.

### Example 2

### Comparison of Protocols for the Depletion or Removal of Compounds (1) and (2) for Determining Cortisol in Human Whole Blood Using Magnetic and Nonmagnetic Mobile Solid-Phase Supports:

### Materials:

Lysed whole blood containing cortisol
Cortisol solution: 250 μg/ml in sterile water
Silica gel beads (magnetic bead mix (20 mg/ml))
Organic solvent mix, 100% acetonitrile
Microtiter plates, UV/Vis-compatible
Microtiter plate reader

### 1) Protocol Using Magnetic Mobile Solid-Phase Supports:

In microtiter plate format, 0, 25, 50 or 75 μl of cortisol solution and 1×, 1.5×, 2× and 4× concentrated silica gel beads (magnetic bead mix solution) are added to 35 μl of whole blood (or 100 μl of serum) in accordance with the following pipetting scheme, thoroughly mixed, and incubated for 1 minute.
Addition of acetonitrile (270 μl in the case of whole blood samples and 360 μl in the case of serum samples) precipitates the proteins at the magnetic beads.
After a 1-minute magnetic separation, the supernatant is taken off, transferred to a free microtiter plate well, and read in the microtiter plate reader at 240 nm.
   2) Protocol without Using Magnetic Mobile Solid-Phase Supports:
1. In microtiter plate format, 0, 25, 50 or 75 μl of cortisol solution and 75, 50, 25 or 0 μl of water are added to 35 μl of whole blood (or 100 μl of serum) in accordance with the pipetting scheme below, thoroughly mixed, and incubated for 1 minute.
2. Addition of acetonitrile (270 μl in the case of whole blood samples and 360 μl in the case of serum samples) precipitates the proteins.
3. The reaction preparation is transferred from microtiter plate format to Eppendorf tube format.
4. The precipitated proteins are collected by a 10-minute centrifugation at 10 000 G and the supernatant is transferred back to microtiter plate format.
5. In the microtiter plate reader, the sample is read at 240 nm.

### Pipetting Scheme:

| | | | Step 3 | | | | |
|---|---|---|---|---|---|---|---|
| | | | add, mix | | | | |
| | | | and | Step 4 | Step 6 | | |
| | | Step 2 | incubate | add and | transfer | Step 7 | |
| | Step 1 | add | 1 min* | mix | the sample | Separation | Step 8 |
| 1 | 35 μl | 0 μl | 100 μl | 270 μl | — | 1-minute | Take off |
| | whole | cortisol | bead sol. | aceto- | | magnetic | super- |
| | blood | solution | (1x conc.) | nitrile | | separation | natant |
| 2 | 35 μl | 25 μl | 75 μl | 270 μl | — | 1-minute | Take off |
| | whole | cortisol | bead sol. | aceto- | | magnetic | super- |
| | blood | solution | (1.5x conc.) | nitrile | | separation | natant |
| 3 | 35 μl | 50 μl | 50 μl | 270 μl | — | 1-minute | Take off |
| | whole | cortisol | bead sol. | aceto- | | magnetic | super- |
| | blood | solution | (2x conc.) | nitrile | | separation | natant |
| 4 | 35 μl | 75 μl | 25 μl | 270 μl | — | 1-minute | Take off |
| | whole | cortisol | bead sol. | aceto- | | magnetic | super- |
| | blood | solution | (4x conc.) | nitrile | | separation | natant |
| 5 | 35 μl | 0 μl | 100 μl | 270 μl | Transfer to | 10-min centri- | Take off |
| | whole | cortisol | water | aceto- | Eppendorf | fugation at | super- |
| | blood | solution | | nitrile | tubes | 10 000 G | natant |
| 6 | 35 μl | 25 μl | 75 μl | 270 μl | Transfer to | 10-min centri- | Take off |
| | whole | cortisol | water | aceto- | Eppendorf | fugation at | super- |
| | blood | solution | | nitrile | tubes | 10 000 G | natant |
| 7 | 35 μl | 50 μl | 50 μl | 270 μl | Transfer to | 10-min centri- | Take off |
| | whole | cortisol | water | aceto- | Eppendorf | fugation at | super- |
| | blood | solution | | nitrile | tubes | 10 000 G | natant |
| 8 | 35 μl | 75 μl | 25 μl | 270 μl | Transfer to | 10-min centri- | Take off |
| | whole | cortisol | water | aceto- | Eppendorf | fugation at | super- |
| | blood | solution | | nitrile | tubes | 10 000 G | natant |

*One-minute incubation for the lysis of the whole blood cells is only necessary for whole blood samples - omitted for serum and water samples.

### Example 3

### Removal of Compounds (2) by MagSi-TDM^{PREP} Mobile Magnetic Solid-Phase Supports Using the Example of Phosphatidylinositol:

### Materials:

Phosphatidyl solution (aqueous): 0.5 g/L
Silica gel beads (magnetic bead mix (50 mg/ml))
Water, analytical purity
Organic solvent mix, 100% acetonitrile
Microtiter plates, UV/Vis-compatible
Microtiter plate reader

### Protocol:

Phosphatidylinositol is dissolved in 0.4 M aqueous potassium bicarbonate, pH 9 solution at a final concentration of 0.5 g/L.
25 μl of phosphatidylinositol solution are added to microcentrifugation tubes.
40 μl of magnetic bead mix are added to the solution and mixed. The solution is then incubated for 30 s.
70 μl of water and 90 μl of acetonitrile are added to the solution.
The silica gel beads are separated in a magnetic field for 1 min.
The supernatant is tested for turbidimetry in the microtiter plate reader at OD₄₀₅.

| Bead type/bead mix | OD₄₀₅ values |
|---|---|
| MagSi-S | 0.1590 |
| MagSi-TDM^{PREP} | 0.1541 |
| MagSi-WCX | 0.3520 |
| MagSi-proteomics C8 | 0.1328 |
| Negative control (without magnetic | 0.3502 |
| beads) | |

### Example 4

### Protocol for Sample Preparation for Whole Blood Samples for the Purpose of LC-MS/MS, but Also Immunoassay; LC or Electrophoresis for the Isolation and Analysis of Trace Components:

Sample material: EDTA blood, heparin blood, citrate blood
1. Lysis solution (double-distilled water) within the range from 100 to 250 μl is added to 50 μl of whole blood, the reaction preparation is mixed and is incubated at room temperature for 30 s-2 min.
2. Optionally, an internal standard, preferably a deuterated internal standard, is added to the reaction preparation in an appropriate concentration, typically in volumes between 10-50 μl. The reaction preparation is then mixed.
3. Optionally, a bead suspension composed of mobile solid-phase supports which may possibly have a specific surface modification, a nonlimiting example being an anion-exchange functionality, can be added to the reaction preparation within the range from 1 mg-10 mg dry weight in a suitable buffer solution. Thereafter, the reaction preparation is incubated at room temperature for 10 s to 2 min. This depletes compounds (2) which cause a matrix effect. Formats that are an alternative to bead suspension can include appropriate filter plates, pipette tips provided with chromatography material (e.g., ZipTips), or columns of differing format.
4. Thereafter, the bead suspension is pelleted in a magnetic field and the supernatant is separated from the mobile solid-phase supports.
5. A further bead suspension in the form of inventive mobile solid-phase supports differing from the bead suspension in (3) in composition, concentration and possibly bead dimensions is added to the supernatant from (4) within the range from 1-10 mg dry weight in a suitable buffer solution.
6. An organic solvent, preferably but not exclusively acetonitrile, is added to the reaction preparation from (5) within the range from ¾ to 3 volume fractions of the reaction preparation, and the reaction preparation is thoroughly mixed. This precipitates proteins and other high-molecular-weight substances at the bead-form mobile solid-phase supports that are present.
7. The magnetic beads together with the precipitate are collected in a magnetic field and the supernatant is taken off.
8. Optionally, the trace components remaining in the supernatant after (7) can be further purified. To this end, the trace components are bound selectively, a nonlimiting example being via an antigen/antibody recognition, or else nonselectively, a nonlimiting example being via a reversed-phase or ion-exchange functionality, to a magnetic bead suspension within the range of 1-10 mg dry weight in a suitable buffer solution, which bead suspension differs from the bead suspension in (5) in composition and concentration. After magnetic separation, the supernatant is discarded and the beads are resuspended in a suitable wash solution. This operation is repeated if necessary and the collected beads are finally gathered in a suitable elution buffer. While doing so, the bound trace components go into solution. Formats that are an alternative to bead suspension can include appropriate filter plates, pipette tips provided with chromatography material (e.g., ZipTips), or columns of differing format.
9. Optionally, compounds (2) remaining in the supernatant can be selectively extracted from the trace components through binding to a bead suspension in the form of mobile solid-phase supports differing from the bead suspension in (5) in composition and concentration, which supports are added within the range of 1-10 mg dry substance in a suitable buffer solution. Formats that are an alternative to bead suspension can include appropriate filter plates, pipette tips provided with chromatography material (e.g., ZipTips), or columns of differing format.
10. Optionally, trace components can be concentrated. To this end, the supernatant from (7) is diluted with water or a polar organic solvent within the range of 2-10 volume fractions of the supernatant, and a bead suspension differing from the bead solution in (5) in composition and concentration is added to the diluted reaction preparation and the trace components are bound. In this case, the binding can proceed similarly to (8) in a selective or nonselective manner. After magnetic separation, the supernatant is discarded and the beads are resuspended in a suitable wash solution. This operation is repeated if necessary and the collected beads are finally gathered in a suitable elution buffer. While doing so, the bound trace components go into solution. Formats that are an alternative to bead suspension can include appropriate filter plates, pipette tips provided with chromatography material (e.g., ZipTips), or columns of differing format.

### Example 5

### Protocol for Sample Preparation for Serum/Plasma for the Purpose of LC-MS/MS, but Also Immunoassay; LC or Electrophoresis for the Isolation or Enrichment and Analysis of Trace Components

Sample material: human serum or plasma
The protocol for sample preparation for serum/plasma samples is identical to the protocol in Example 4—whole blood samples—differing in that step (1) from Example 4 is omitted.

### Example 6

### Protocol for Sample Preparation for Whole Blood Samples for the Purpose of LC-MS/MS, but Also Immunoassay; LC or Electrophoresis for the Isolation and Analysis of Trace Components (Salt Lysis):

1. 15 μl of lysis solution—aqueous ZnSO₄ (analytical purity) are added to 50 μl of whole blood within the range of 0.2 M-1 M and incubated at room temperature for 0.5-2 min.
   All further steps follow Example 4, steps 2 to 10.

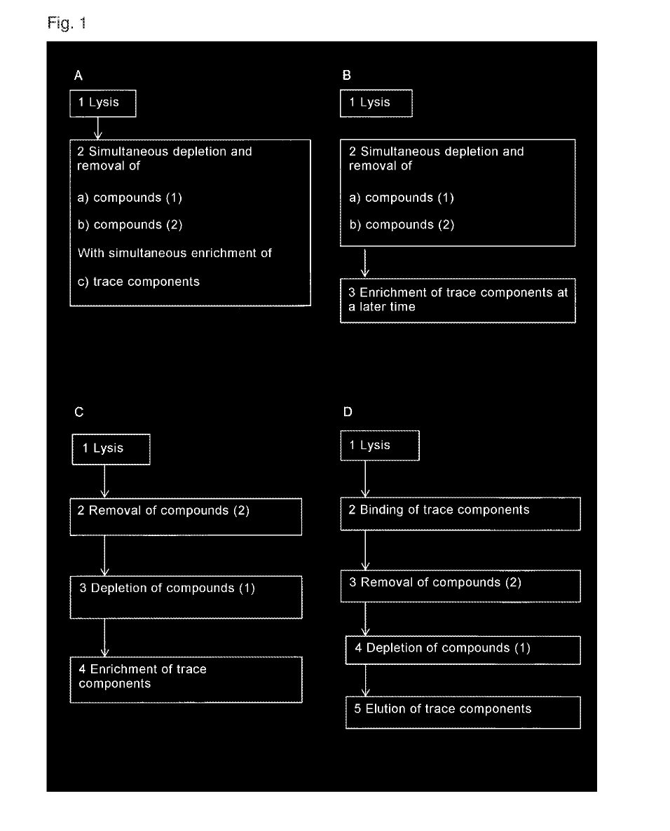

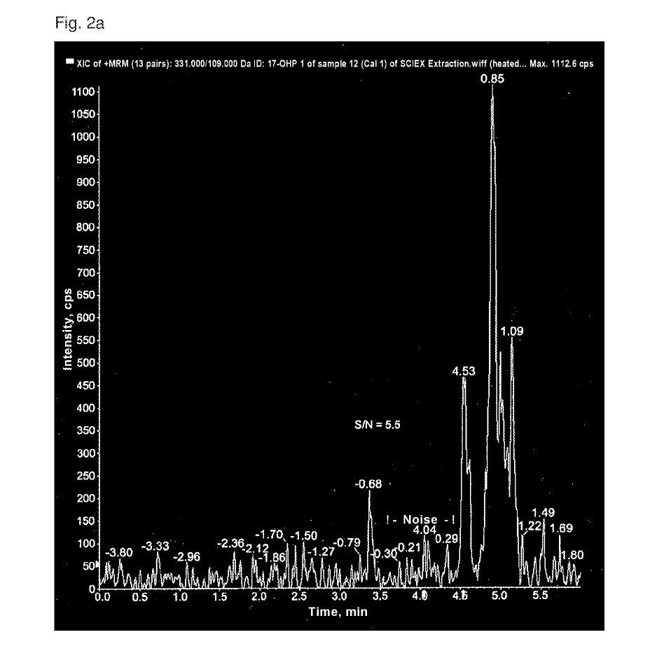

The method and kit according to the disclosure will be explained again with the aid of FIG. 1, which follows:

In the method according to the disclosure and in the method for isolating trace components with the aid of the kit, it is possible to carry out individual process steps simultaneously or separately, in one or more cavities. FIG. 1 shows possible process steps covered by the present disclosure. The depletion or removal of compounds (1) and (2) can take place simultaneously (cases A and B) or separately (cases C and D). An isolation of a trace component (if necessary) usually, but not exclusively, takes place as the last process step. In the case of whole blood samples, the lysis of red blood cells is a necessary process step—in the case of other clinical samples such as serum/plasma or saliva, this process step can be omitted.

### Example 7

### Comparative Measurements of the Serum Concentration of Trace Components Using Four Different Protocols

### Materials

LC-MS/MS system
Protocol 1: Protein Precipitation at Silica Gel Beads (Magnetic Beads) with Subsequent Magnetic Separation
1. 20 μl of internal standard and 40 μl of silica gel bead solution are added to 50 μl of human serum containing metabolite analyte and mixed.
2. 130 μl of acetonitrile as precipitation agent are added to the reaction preparation and mixed vigorously.
3. The reaction preparation is separated on a magnetic separator for 1 minute and 80 μl of the clear supernatant are transferred to an HPLC vessel.

Sample preparation time per sample: approx. 2 minutes

Protocol 1 is especially suitable for analytes present in serum in comparatively high concentrations. These include: antibiotics, benzodiazepines, antidepressants, neuroleptics.

### Protocol 2: Typical “Protein-Crash” Protocol for Rapid Sample Preparation

1. 200 μl of methanol already containing the internal standard are added to 200 μl of human serum and mixed vigorously.
2. The reaction preparation is centrifuged at 5000 g for 10 min.
3. 80 μl of the clear supernatant are transferred to an HPLC vessel.

Sample preparation time per sample: approx. 12 min

Protocol 2 is especially suitable for trace elements present in serum in comparatively low concentrations. These include: catecholamines, steroid hormones, prostaglandins. Furthermore, protocol 2 is suitable for when the reaction preparation has to be adjusted after sample preparation to the LC conditions (rebuffering).

Protocol 3: Liquid-Liquid Extraction without Silica Gel Beads (or without Magnetic Beads)
1. A mixture of ethyl acetate, dichloromethane and isopropanol (ratio 3:1:1) is added to 200 μl of human serum containing metabolite analyte and mixed vigorously.
2. Incubation for 2 minutes for the purpose of separation of the aqueous phase and organic phase.
3. 300 μl of the organic phase are transferred to an HPLC vessel.
4. The organic phase is vaporized in a stream of nitrogen until dry and reconstituted in 100 μl of methanol.

Sample preparation time per sample: approx. 12-17 min

### Protocol 4: Protein Precipitation at Silica Gel Beads (Magnetic Beads) Followed by Liquid/Liquid Extraction

1) 20 μl of internal standard and 160 μl of silica gel beads are added to 200 μl of human serum and mixed.
2) 520 μl of acetonitrile as precipitation agent are added to the reaction preparation and mixed vigorously.
3) The reaction preparation is separated on an M12+12 magnetic separator (MagnaMedics) for 1 minute and 300 μl of the clear supernatant are transferred to an HPLC vessel.
4) A mixture of ethyl acetate, dichloromethane and isopropanol (ratio 3:1:1) is added to the supernatant and mixed vigorously.
5) 300 μl of the organic phase are transferred to an HPLC vessel.
6) The organic phase is vaporized in a stream of nitrogen until dry and reconstituted in 100 μl of methanol.

| Analyte | Protocol | Area | IS area |
|---|---|---|---|
| Arachidonic acid | Magnetic beads | 3736009 | 130817 |
| | direct | | |
| | Centrifugation | 264096 | 8310 |
| | Liquid/liquid | 2137996 | 60391 |
| | extraction | | |
| | Magnetic | 3458125 | 100706 |
| | beads/liquid-liquid | | |
| | extraction | | |
| 5-HETE | Magnetic beads | 660739 | 2578 |
| | direct | | |
| Prostaglandin | Centrifugation | 165336 | 704 |
| | Liquid/liquid | 304197 | 1141 |
| | extraction | | |
| | Magnetic | 398497 | 1432 |
| | beads/liquid-liquid | | |
| | extraction | | |

Area or IS area refers to the region below the signal peak.

### Example 8

### Comparative Measurements Between Magnetic Silica Gel Bead Sample Preparation Method and a Simple “Protein-Crash” Method—Adjustment of the Reaction Preparation to the Mobile Phase

### Materials:

LC-MS/MS system

### LC

2 ml Eppendorf tubes

### Magnetic Separator

Protocol 1: Protein Precipitation at Magnetic Silica Gel Beads with Subsequent Magnetic Separation
1. 20 μl of internal standard and a mixture of 40 μl of magnetic silica gel bead solution and 10 μl of aqueous zinc sulfate magnetic silica gel bead solution and 10 μl of aqueous zinc sulfate (2 M) are added to 50 μl of a human serum containing steroid hormone analyte and mixed.
2. 250 μl of methanol as precipitation agent are added to the reaction preparation and mixed vigorously.
3. The reaction preparation is separated on a magnetic separator for 1 minute and 50 μl of the clear supernatant is transferred to an HPLC vessel.
4. 25 μl of double-distilled H₂O are added to the supernatant and mixed well (optional step). The pure water acts here as a correction solution for the mobile phase.
5. The resultant optimized water:methanol ratio of near 1:1 optimizes the LC-MS/MS chromatogram significantly.

Sample preparation time per sample: approx. 2 minutes

Protocol 1 is especially suitable for analytes present in serum in comparatively high concentrations. If the protein precipitation conditions (proportion of organic phase) and LC conditions (composition of the mobile phase) are far apart, the optional addition of the correction solution is carried out as described above. Here are typical trace components: steroid hormones, such as cortisol, cortisone, deoxycortisol, deoxycorticosterone, corticosterone, androsterone, progesterone, pregnenolone, estrogen, estrone, estradiol, estriol, testosterone, 17-hydroxyprogesterone, antibiotics, polar vitamins such as vitamins B1 and B6.

### Protocol 2:

1. 200 μl of methanol containing the internal standard are added to 200 μl of a human serum and mixed vigorously.
2. The reaction preparation is centrifuged at 5000 g for 10 min.
3. 75 μl of the clear supernatant is transferred to an HPLC vessel.

Protocol 2 is especially suitable for when the precipitation conditions and the LC conditions (composition of the mobile phase) are close together. It is then possible to omit the optional step of adding double-distilled H₂O. Here are typical trace components: apolar vitamins A, E and K.

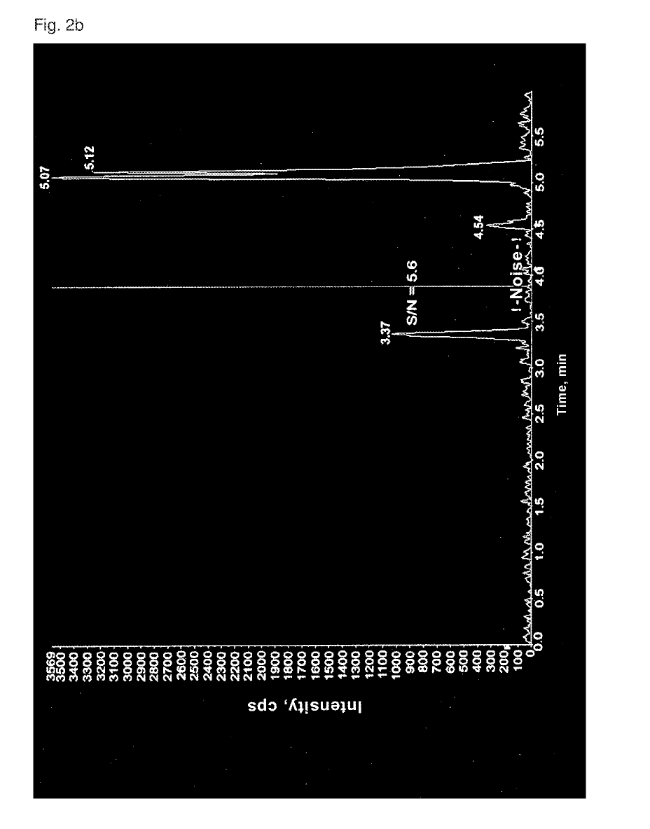

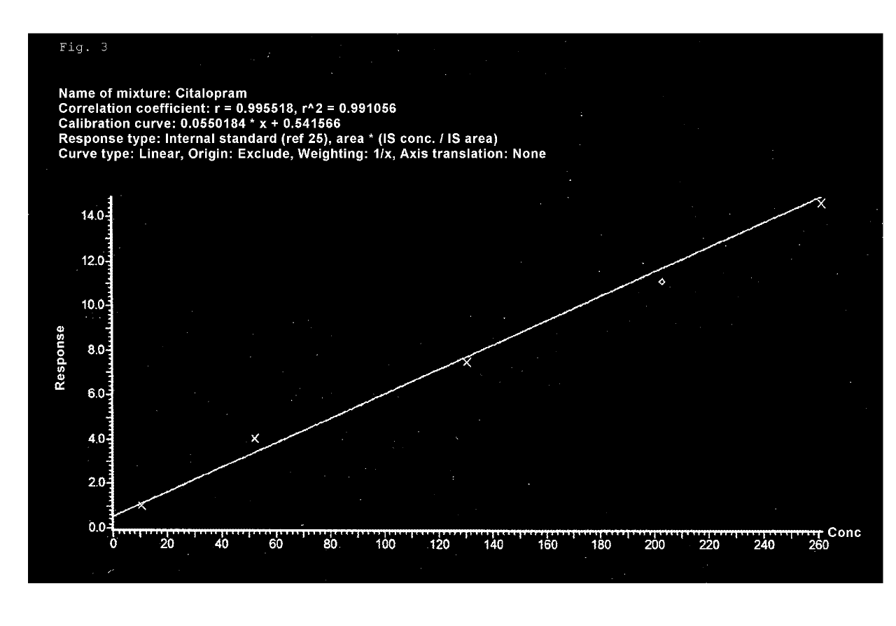

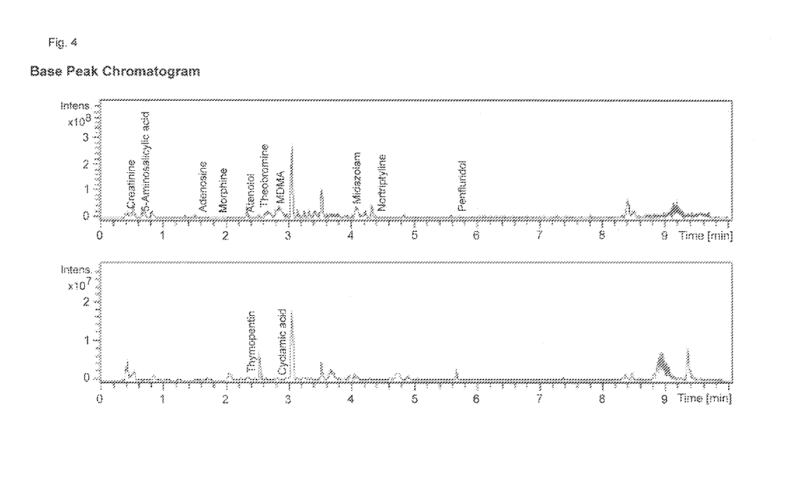

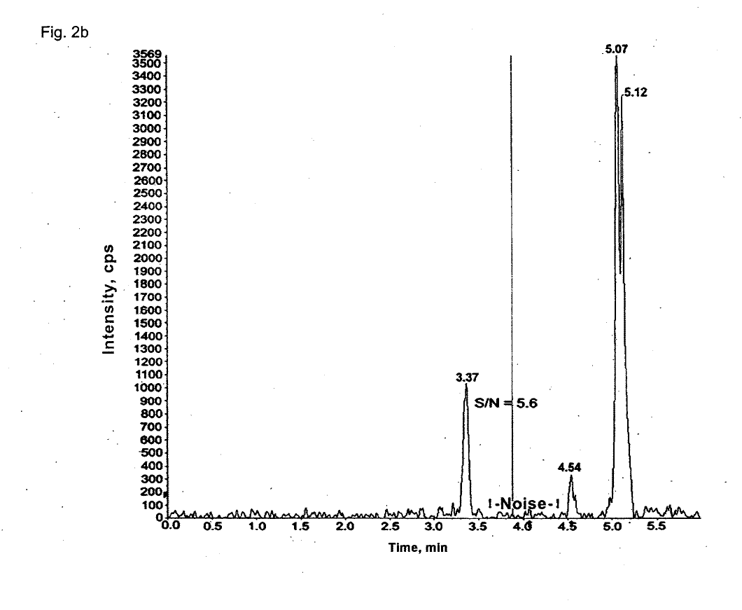

The effect of a sample preparation according to Example 8 for protocols 1 and 2 is depicted in FIGS. 2a and 2b for 17-hydroxyprogesterone, where FIG. 2b depicts the sample preparation according to protocol 1 and FIG. 2a depicts the sample preparation according to protocol 2. The background noise can be distinctly reduced in protocol 1 in comparison with protocol 2.

### Example 9

Multiplex Assays from Urine

### Materials:

2 ml Eppendorf tubes
Magnetic separator

### Protocol 1

1. 20 μl of internal standard, 20 μl of urine stabilization buffer and 40 μl of MagSi-TOX^{PREP} bead mix are added to 50 μl of urine and mixed.
2. 130 μl of acetonitrile are added thereto and mixed vigorously.
3. The reaction preparation is separated on a magnetic separator for 1 minute and 80 μl of the clear supernatant are transferred to an HPLC vessel.

| Parameter | Parameter class | Matrix effect in % |
|---|---|---|
| Amphetamine | Drug abuse | 103 |
| Methamphetamine | Drug abuse | 93 |
| MDMA | Drug abuse | 80 |
| MDA | Drug abuse | 78 |
| 7-Aminoflunitrazepam | Benzodiazepine | 84 |
| Lorazepam | Benzodiazepine | 91 |
| Oxazepam | Benzodiazepine | 101 |
| Temazepam | Benzodiazepine | 100 |
| Nordiazepam | Benzodiazepine | 96 |
| Diazepam | Benzodiazepine | 106 |
| Methadone | Drug abuse | 123 |
| Bromazepam | Benzodiazepine | 93 |

Protocol 1 is especially suitable for compounds which are typically screened in multiparameter assays. These include in particular: drug abuse analysis, benzodiazepine analysis, newborn screening, pain management.

For example, benzodiazepines such as clonazepam, nitrazepam, diazepam

### Example 10

### Whole Blood Cell Lysis

### Materials

Light microscope
Slide and coverslip

### Double-distilled H₂O

Whole blood

### Protocol 1

The rate of hemolysis of red blood cells can be carried out directly under a transmitted light microscope at a magnification.
1 40 μl, 50 μl, 60 μl, 70 μl, 80 μl, 90 μl and 100 μl of double-distilled H₂O are added to 25 μl of whole blood, mixed, and incubated at room temperature for 1 minute.
2. 2 μl of the reaction mixture are transferred to a slide and are analyzed under the transmitted light microscope for intact red blood cells that are present.

| | Water volume | | | | | | |
|---|---|---|---|---|---|---|---|
| | 40 μl | 50 μl | 60 μl | 70 μl | 80 μl | 90 μl | 100 μl |
| Intact | yes | no | no | no | no | no | no |
| cells | | | | | | | |
| present | | | | | | | |

2 volumes of water to 1 volume of whole blood was determined to be the minimum rate of pure water as haemolytic reagent. Owing to this ratio, viscosity is additionally lowered to an extent which ensures an optimal magnetic separation of silica gel beads using the method according to the disclosure.

The parameters determined from lysed whole blood include: immunosuppressants, combined with vitamin B1/B6.

### Example 11

Determination of immunosuppressant concentrations from human whole blood. Analysis of critical factors during the sample preparation.

### Materials:

2 ml Eppendorf tubes
Magnetic separator
LC-MS/MS system with appropriate analysis software
Whole blood samples

### Protocol 1:

1. 60 μl of double-distilled H₂O are added to 25 μl of whole blood for the purpose of lysis of whole blood cells, mixed, and incubated for 1 minute.
2. 10 μl of internal standard mix solution in methanol are added to the reaction preparation. Here, the internal standard mix should consist of deuterated cyclosporine A, deuterated tacrolimus, deuterated sirolimus and deuterated everolimus.
3. 40 μl of silica bead mix (50 mg/mL) or a mixture of 40 μl of silica gel beads ((bead mix (50 mg/mL)) and 10 μl of aqueous zinc sulfate heptahydrate solution (2 M) are added to the reaction preparation.
4. x μl of acetonitrile (ACN) are added to the reaction preparation and mixed vigorously. The mix is either pipetted up and down 10× or the reaction preparation is vortexed.
5. In a suitable magnet, the silica gel beads (bead mix/protein precipitate mixture) are separated magnetically within one minute.
6. 50 μl of supernatant are taken off and transferred to a suitable vessel, e.g., HPLC glass vials.
7. The thus processed sample is measured in the LC-MS/MS and the data are evaluated using appropriate analysis software.

| | Precip- | | | | |
|---|---|---|---|---|---|
| | itation | Area | | | |
| | volume | under the | | | |
| | (aceto- | substance | | Area/ | |
| Sample | nitrile) | peak | Area IS | Area IS | Adaptations |
| PBS | ACN, | 2696 | 6142 | 0.4390 | Mixing by |
| buffer | 190 μl | | | | pipetting |
| PBS | ACN, | 2701 | 5711 | 0.4729 | Mixing by |
| buffer | 190 μl | | | | pipetting |
| Whole | ACN, | 913 | 1319 | 0.6925 | Mixing by |
| blood | 190 μl | | | | pipetting |
| Whole | ACN, | 807 | 1239 | 0.6515 | Mixing by |
| blood | 190 μl | | | | pipetting |
| Whole | ACN, | 1233 | 2559 | 0.4819 | Additional |
| blood | 190 μl | | | | precipitation |
| | | | | | with ZnSO₄ |
| Whole | ACN, | 1082 | 1964 | 0.5509 | Additional |
| blood | 190 μl | | | | precipitation |
| | | | | | with ZnSO₄ |
| Whole | ACN, | 607 | 990 | 0.6124 | Mixing by |
| blood | 190 μl | | | | vortexing |
| Whole | ACN, | 584 | 951 | 0.6136 | Mixing by |
| blood | 190 μl | | | | vortexing |
| Whole | ACN, | 928 | 2405 | 0.3859 | Mixing by |
| blood | 190 μl | | | | vortexing |
| Whole | ACN, | 1010 | 2768 | 0.3650 | Mixing by |
| blood | 190 μl | | | | vortexing |
| Whole | ACN, | 1055 | 2357 | 0.4478 | Mixing by |
| blood | 220 μl | | | | pipetting |
| Whole | ACN, | 1077 | 2326 | 0.4631 | Mixing by |
| blood | 220 μl | | | | pipetting |
| Whole | ACN, | 1189 | 2756 | 0.4312 | Mixing by |
| blood | 250 μl | | | | pipetting |
| Whole | ACN, | 1277 | 3207 | 0.3982 | Mixing by |
| blood | 250 μl | | | | pipetting |
| Whole | ACN, | 1135 | 2803 | 0.4050 | Mixing by |
| blood | 280 μl | | | | pipetting |
| Whole | ACN, | 1157 | 2703 | 0.4281 | Mixing by |
| blood | 280 μl | | | | pipetting |
| Whole | ACN, | 861 | 2372 | 0.3631 | Additional |
| blood | 220 μl | | | | precipitation |
| | | | | | with ZnSO₄ |
| Whole | ACN, | 602 | 1690 | 0.3563 | Additional |
| blood | 220 μl | | | | precipitation |
| | | | | | with ZnSO₄ |
| Whole | ACN, | 523 | 1267 | 0.4126 | Additional |
| blood | 250 μl | | | | precipitation |
| | | | | | with ZnSO₄ |
| Whole | ACN, | 506 | 1356 | 0.3732 | Additional |
| blood | 250 μl | | | | precipitation |
| | | | | | with ZnSO₄ |
| Whole | ACN, | 454 | 1373 | 0.3307 | Additional |
| blood | 280 μl | | | | precipitation |
| | | | | | with ZnSO₄ |

### Example 12

Positive Selection of Vitamin D—Binding of Trace Components to Silica Gel-Based Surfaces Such as Solid-Phase Supports for the Purpose of Isolation or Concentration and/or Additionally Purification Following Protocol 1 in Relation to Example 12

### Materials:

Vitamin D as powder
Microtiter plate
Silica gel beads (bead mix (x mg/ml))

### Acetonitrile

### Double-distilled H₂O

UV/Vis reader

### Protocol 1

1. 80 μl of sample (vitamin D, 16 nmol, in 60% aqueous acetonitrile, similar to the solution composition following the sample preparation from protocol 1) are initially charged into a deep-well microtiter plate for 96 samples.
2. 250 μl of diluted bead mix are added to the sample and incubated at room temperature for 8 min. During the incubation, the reaction preparation is mixed twice by pipetting. The diluted bead mix simultaneously serves as binding buffer.
3. The microtiter plate is transferred to a suitable magnet and the magnetic beads are exposed to the magnetic separator for 1 minute.
4. The magnetic beads are washed 1× with 100 μl of pure water.
5. The magnetic beads are resuspended in 30 μl of desorption solution (80% aqueous acetonitrile) and incubated for 30 seconds.
6. Removal of the magnetic silica gel beads (bead mix for 1 minute).
7. 25 μl of the supernatant are taken off and transferred to a fresh microtiter plate.
8. The purified sample is diluted with 75 μl of pure water and analyzed in the UV/Vis reader.
9. To this end, an absorption spectrum is recorded between A230-A320 nm and the concentration is determined from the area under the peak.

| | Vitamin D quantity | Vitamin D quantity in |
|---|---|---|
| | in binding buffer | desorption solution |
| Bead | [nmol] | [nmol] |
| MagSi-proteomics C18 | 2.5 | 16.2 |
| MagSi-C18 ferrofluid | 1 | 18.6 |
| Dynal RPC | 3.9 | 12.5 |
| MagSi-proteomics C8 | 2.3 | 17.8 |
| MagSi-Phenyl-A | 1.4 | 16.8 |
| MagSi-Phenyl-E | 1.8 | 15.4 |
| MagSi-C18_endcapped | 0 | 15.1 |

### Example 13

### Drug Abuse Analysis Using the Example of Ethyl Glucuronide

### Materials

LC-MS/MS system with appropriate analysis software
Human serum
Magnetic separator

### Protocol 1

a) 20 μl of internal standard and 40 μl of magnetic silica gel bead solution are added to 50 μl of human serum containing ethyl glucuronide and mixed.
b) 130 μl of acetonitrile as precipitation reagent are added to the reaction preparation and mixed vigorously.
c) The reaction preparation is separated on the magnetic separator for 1 minute and 80 μl of the clear supernatant are transferred to an HPLC vessel and analyzed.

### Example 14

### Anticoagulants Apixaban

### Materials:

Silica gel beads (bead mix MagSi-TDM^{PREP} type 1 and type 2)
LC-MS/MS system with appropriate analysis software
Human serum
Magnetic separator

### Protocol 1

a) 20 μl of internal standard and 40 μl of magnetic bead solution type 1 or type 2 are added to 50 μl of human serum containing ethyl glucuronide and mixed.
b) 130 μl of acetonitrile as precipitation reagent are added to the reaction preparation and mixed vigorously.
c) The reaction preparation is separated on the magnetic separator for 1 minute and 80 μl of the clear supernatant are tranferred to an HPLC vessel.

| | Response at 3 μg/l | | |
|---|---|---|---|
| | ACN protein | Bead mix MagSi- | Bead mix MagSi- |
| | crash | TDM type 1 | TDM type 2 |
| | 1229772 | 1329533 | 1303946 |
| | 1409106 | 1287669 | 1290592 |
| | 1491590 | 1390365 | 1307791 |
| | 1513081 | 1296862 | 1247809 |
| | 1608383 | 1364863 | 1180736 |
| | 1462602 | 1320643 | 1436992 |
| | 1648652 | 1530894 | 1275473 |
| Mean | 1480455 | 1360118 | 1291906 |
| Standard | 137898 | 83512 | 77484 |
| deviation | | | |
| Precision | 9.31 | 6.14 | 6.00 |

Wherein a defined precision=(standard deviation/mean)×100 is assumed, i.e., the lower the value, the better the result. Therefore, in the present example, the method using silica gel bead solution MagSi-TDM^{PREP} TYPE 2 from MagnaMedics appears to be best suited to the isolation of the present trace components.

### Example 15

### Thaw Stability of Amiodarone in Serum

### Materials:

Silica gel beads (bead mix MagSi-TDM^{PREP} type 1 and type 2)
LC-MS/MS system with appropriate analysis software
Human serum
Magnetic separator

### Protocol 1

1. 20 μl of internal standard and 40 μl of magnetic silica gel bead solution type 1 or type 2 are added to 50 μl of human serum containing amiodarone and mixed.
2. 130 μl of acetonitrile as precipitation reagent are added to the reaction preparation and mixed vigorously.
3. The reaction preparation is separated on the magnetic separator for 1 minute and 80 μl of the clear supernatant are transferred to an HPLC vessel.
4. After each measurement, the processed serum sample was frozen at 18° C. and rethawed.

| Amiodarone | QC 1 | | QC 2 | | QC 3 | |
|---|---|---|---|---|---|---|
| thaw | (1.096 | Recov- | (1.644 | Recov- | (2.740 | Recov- |
| stability | mg/L) | ery | mg/L) | ery | mg/L) | ery |
| Intra-assay | 1.1 | | 1.7 | | 2.7 | |
| 1x thaw | 1.1 | 100.0% | 1.7 | 100.0% | 2.7 | 100.0% |
| 2x thaw | 1.1 | 100.0% | 1.7 | 100.0% | 3.1 | 114.8% |
| 3x thaw | 1.2 | 109.1% | 1.7 | 100.0% | 2.8 | 103.7% |

QC = quality control with 3 different values QC 1, QC 2 and QC 3.

Protocol 1 is especially suitable for the analysis of cardiovascular and antiarrhythmic trace elements from human serum samples.

### Example 16

Analysis of steroids

### Materials:

Silica gel beads (bead mix MagSi-TDM^{PREP} type 1 and type 2), LC-MS/MS system with appropriate analysis software, human serum, magnetic separator

### Protocol 1

1. 20 μl of internal standard and 40 μl of a magnetic silica gel bead type 1 or type 2 are added to 50 μl of human serum and mixed.
2. 130 μl of acetonitrile as precipitation reagent are added to the reaction preparation and mixed vigorously.
3. The reaction preparation is separated on the magnetic separator for 1 minute and 80 μl of the clear supernatant are transferred to an HPLC vessel.

| Recovery | QC 1 | QC 2 | QC 3 |
|---|---|---|---|
| (μg/l) | (230.6) | (577.0) | (1153) |
| Mean value | 239.5 | 582.2 | 1176.7 |
| (n = 24) | | | |
| Nonextracted | 241.2 | 549.1 | 1165.4 |
| standard | | | |
| Recovery | 99.3% | 106.0% | 101.0% |
| | QC 1 | Q 2 | Q 3 |
| | (230.6 | (577.0 | (1153 |
| Accuracy | ng/ml) | ng/ml) | ng/ml) |
| Intra-assay | 240.7 | 613.5 | 1160.7 |
| (n = 5, | | | |
| ng/ml) | | | |
| | 250.4 | 580.9 | 1176.3 |
| | 252.4 | 610.9 | 1244.1 |
| | 235.5 | 594.9 | 1133.3 |
| | 251.9 | 614.4 | 1254.2 |
| Mean | 246.2 | 602.9 | 1193.7 |
| Accuracy % | 106.8% | 104.5% | 103.5% |
| Standard | 7.6 | 14.6 | 53 |
| deviation | | | |
| CV % | 3.1% | 2.4% | 4.4% |

QC = quality control with 3 different values QC 1, QC 2 and QC 3

The above protocol is thus especially suitable for the analysis of dexamethasone, mineralocorticoids, stereoid hormones.

### Example 17

Antidepressant Analysis from Serum

### Materials:

Silica gel beads (bead mix MagSi-TDM^{PREP} type 1 and type 2)
LC-MS/MS system with appropriate analysis software
Human serum
Magnetic separator

### Protocol 1

1. 20 μl of internal standard and 40 μl of bead mix MagSi-TDM^{PREP} type 1 or type 2 are added to 50 μl of human serum and mixed.
2. 130 μl of acetonitrile as precipitation reagent are added to the reaction preparation and mixed vigorously.
3. The reaction preparation is separated on the magnetic separator for 1 minute and 80 μl of the clear supernatant are transferred to an HPLC vessel.

FIG. 3 depicts by way of example the calibration curve for the antidepressant under investigation using the example of citalopram. The linear profile of the regression lines for immunoassays and LC-MS/MS can be seen. The virtually linear profile over the entire range of analysis allows a high reproducibility of the results and a high likelihood of recovery of the trace components under investigation or compounds 1 and/or 2.

### Example 18

### Urine Analysis

Materials: Silica gel beads (bead mix MagSi-TOX^{PREP} type 1 and type 2), LC-MS/MS system with appropriate analysis software, human serum, magnetic separator

### Protocol 1

20 μl of internal standard mix, 20 μl of urine stabilization buffer and 40 μl of bead mix MagSi-TOX^{PREP} type I or type II are added to 50 μl of urine and mixed.
130 μl of acetonitrile are added thereto and mixed vigorously.
The reaction preparation is separated on the magnetic separator for 1 minute and 80 μl of the clear supernatant are transferred to an HPLC vessel.

To illustrate the substances mentioned by way of example in the following table for the urine analysis, FIG. 4 depicts by way of example the chromatogram of the substances from a urea-containing solution such as, for example, human urine that are determined using the method according to the disclosure.

| Parameter | Parameter class | Intensity in units |
|---|---|---|
| Caffeine | Anti-asthma | 16 |
| Theophylline | Anti-asthma | 13 |
| Midazolam | Benzodiazepines | 7.4 |
| Clozapine | Neuroleptic | 7.3 |
| | medicaments | |
| 6-O-Acetylmorphine | Drug abuse | 6.8 |
| Atenolol | Antiarrythmic | 6.2 |
| Nortriptyline | Tricyclic | 6.1 |
| | antidepressants | |
| Theobromine | Alkaloid | 4.8 |
| Oxycodone | Analgesic | 4 |
| Adenosine | Antagonist of | 1.6 |
| | catecholamines | |
| 5-Aminosalicylic acid | Anti-inflammatory | 2.2 |
| MDMA | Drug abuse | 1.4 |

1. A method for isolating trace components selected from the group of cardiovascular substances, immunosuppressants, substances for treating the central nervous system, antibiotics, antimycotics, antidepressants, neuroleptics, tricyclic antidepressants, benzodiazepines, antidepressants, antibiotics, psychopharmaceuticals, cardiovascular medicaments, drugs, insecticides, pesticides, fungicides, antibodies, vasopressin, oxytocin, somatostatin, enkephalin, beta-endorphin, thyroxine, triiodothyronine, thyroglobulin, calcitonin, catecholamine, dopamine, serotonin, parathormone, aldosterone, renin, angiotensin, cortisol, cortisone, deoxycortisol, deoxycorticosterone, corticosterone, androsterone, progesterone, pregnenolone, estrogen, estrone, estradiol, estriol, testosterone, FSH, gonadotropin, cyclosporine A, tacrolimus, everolimus, sirolimus, MPA, insulin, anti-insulin antibodies, glucagon, gastrin, secretin, AMP, GMP, MMP 20, ZAP 1, ZAP 2, ZAP 3, prostaglandins, thromboxane, erythropoietin, histamine, phenobarbital, phenytoin, carbamazepine, primidone, ethosuximide, valproic acid, acetazolamide, sultiame, glutethimide, clonazepam, nitrazepam, diazepam, pentobarbital, secobarbital, bupivacaine, mepivacaine, lidocaine, procainamide, quinidine, digoxin, digitoxin, theophylline, amitriptyline, imipramine, amikacin, gentamicin, tobramycin, cefalexin, sulfamethoxazole, methotrexate, cyclosporine, methylprednisolone, salicylic acid, acetaminophen, indomethacin, allopurinol, vitamin A, carotene, vitamin B1; vitamin B2; vitamin B6; folic acid, vitamin C, vitamin D, 25-OH vitamin D3, 25-OH vitamin D2, vitamin E, angiotensin I and II, hemopexin, transferrin, cortisol, insulin, fibrinogen, antithrombin, plasminogen, antiplasmin, methylmalonic acid, folic acid, arachidonic acid, prostaglandin E2 and homocysteine from a liquid biological sample for sample preparation in chemical analysis and diagnostics by means of a purification process for proteins, peptides, amino acids, sugars, lipids, phospholipids and inorganic salts, comprising:
a) lysis of the cells and/or
b) depletion of proteins by binding on mobile, magnetic silica gel beads and/or
c) removal of peptides, amino acids, sugars, lipids, phospholipids and inorganic salts by binding on mobile, magnetic silica gel beads,
wherein the binding on the mobile, magnetic silica gel beads under b) and/or c) can take place non-selectively, selectively and/or reversibly,
wherein the isolation of the trace components takes place timely offset after steps b) and/or c) with the aid of magnetic separation by binding on the surface of the mobile, magnetic silica gel beads, which differ from the mobile, magnetic silica gel beads under b) and/or c) in terms of different functional groups.

2. The method as claimed in claim 1, wherein a binding of proteins, peptides, amino acids, sugars, lipids, phospholipids, inorganic salts and trace components on the mobile, magnetic silica gel beads is achieved by protic or aprotic solvents and proteins, peptides, amino acids, sugars, lipids, phospholipids, inorganic salts and/or trace components become eluted.

3. The method as claimed in claim 2, wherein the eluents encompass hexane, acetonitrile, methanol, ethanol, H₂O, isopropanol, n-propanol, isobutanol, n-butanol and DMSO.

4. The method as claimed in claim 1, wherein the depletion of proteins, peptides, amino acids, sugars, lipids, phospholipids, inorganic salts and/or the isolation of trace components is performed on mobile, magnetic silica gel beads having a bead diameter between 100 nm and 100 μm.

5. The method as claimed in claim 4, wherein the surfaces of the beads include functional groups selected from the group consisting of, for example, OH, —COOH, NH₂, R—SO₂—OH, —NH₂; —RNH, —R₂N, —CH₃, —C₂H₅, —C₄H₉, —C₈H₁₇, —C₆H₅, —ZrO₂, TiO₂, C₆H₉NO₆, phenylhexyl, biphenyl, hydroxyapatite and boronic acid.

6. The method as claimed claim 1, wherein the lysis under step a) includes the following steps:
aa) 1.5 to 10 volumes of a buffer solution of low ionic strength having a salt concentration of 0-0.5 M are added to one volume of a biological material, or
bb) 0.005 to 2.5 parts by volume of the buffer of a high ionic strength are added to one part by volume of a biological liquid, which includes a divalent salt having a concentration within the range from 0.1 M to 3 M.

7. The method as claimed claim 1, wherein the trace components, especially vitamin D, are bound on the mobile, magnetic silica gel beads and become eluted by varying the polarity of the solvent and/or the pH and/or the salt concentration.

8. The method as claimed in claim 7, wherein the binding of the proteins, peptides, amino acids, sugars, lipids, phospholipids, inorganic salts and the isolation of the trace components is achieved by a change in the dielectric constant by addition of at least one organic solution which has a dipole moment between 1.6 and 4.0 Debye at a volume fraction within a range between 1.5 and 4.

9. The method as claimed in claim 1, wherein an isolation of the trace components is achieved by binding with the mobile, magnetic silica gel beads by means of specific interactions, comprising:
a) ionic interactions with respect to the binding of the charged compounds,
b) hydrogen bonds with respect to the binding of the polar compounds,
c) hydrophobic interactions with respect to the binding of the hydrophobic compounds,
d) π-π electron interactions with respect to the binding of compounds containing delocalized π electron systems such as aromatics,
e) metal affinity bonds with respect to the binding of selected compounds such as phosphate groups.

10. The method as claimed in claim 1, wherein the remaining trace components are separated by magnetic fields and/or centrifugation and analyzed by electrophoresis, CE, HPLC, UPLC, LC/MS, LC-MS/MS, UV/Vis, immunoassay detection or flow cytometry.

11. Trace components from a liquid biological sample for sample preparation in chemical analysis and diagnostics selected from a group of cardiovascular substances, immunosuppressants, substances for treating the central nervous system, antibiotics, antimycotics, antidepressants, neuroleptics, tricyclic antidepressants, benzodiazepines, antidepressants, antibiotics, psychopharmaceuticals, cardiovascular medicaments, drugs, insecticides, pesticides, fungicides, antibodies, vasopressin, oxytocin, somatostatin, enkephalin, beta-endorphin, thyroxine, triiodothyronine, thyroglobulin, calcitonin, catecholamine, dopamine, serotonin, parathormone, aldosterone, renin, angiotensin, cortisol, cortisone, deoxycortisol, deoxycorticosterone, corticosterone, androsterone, progesterone, pregnenolone, estrogen, estrone, estradiol, estriol, testosterone, FSH, gonadotropin, cyclosporine A, tacrolimus, everolimus, sirolimus, MPA, insulin, anti-insulin antibodies, glucagon, gastrin, secretin, AMP, GMP, MMP 20, ZAP 1, ZAP 2, ZAP 3, prostaglandins, thromboxane, erythropoietin, histamine, phenobarbital, phenytoin, carbamazepine, primidone, ethosuximide, valproic acid, acetazolamide, sultiame, glutethimide, clonazepam, nitrazepam, diazepam, pentobarbital, secobarbital, bupivacaine, mepivacaine, lidocaine, procainamide, quinidine, digoxin, digitoxin, theophylline, amitriptyline, imipramine, amikacin, gentamicin, tobramycin, cefalexin, sulfamethoxazole, methotrexate, cyclosporine, methylprednisolone, salicylic acid, acetaminophen, indomethacin, allopurinol, vitamin A, carotene, vitamin B1; vitamin B2; vitamin B6; folic acid, vitamin C, vitamin D, 25-OH vitamin D3, 25-OH vitamin D2, vitamin E, angiotensin I and II, hemopexin, transferrin, cortisol, insulin, fibrinogen, antithrombin, plasminogen, antiplasmin, methylmalonic acid, folic acid, arachidonic acid, prostaglandin E2 and homocysteine received by means of a purification process for proteins, peptides, amino acids, sugars, lipids, phospholipids and inorganic salts wherein
a) a lysis of the cells and/or
b) a depletion of proteins by binding on mobile, magnetic silica gel beads and/or
c) a removal of peptides, amino acids, sugars, lipids, phospholipids and inorganic salts by binding on mobile, magnetic silica gel beads,
takes place and wherein the binding on the mobile, magnetic silica gel beads under b) and/or c) takes place non-selectively, selectively and/or reversibly,
wherein the isolation of the trace components takes place timely offset after steps b) and/or c) with the aid of magnetic separation by binding on the surface of the mobile, magnetic silica gel beads, which differ from the mobile, magnetic silica gel beads under b) and/or c) in terms of different functional groups.

12. The trace components received by means of a purification process for proteins, peptides, amino acids, sugars, lipids, phospholipids and inorganic salts according to claim 11 wherein the trace components become eluted by eluents encompassing hexane, acetonitrile, methanol, ethanol, H₂O, isopropanol, n-propanol, isobutanol, n-butanol and DMSO.

13. The trace components received by means of a purification process for proteins, peptides, amino acids, sugars, lipids, phospholipids and inorganic salts according to claim 11 wherein the depletion of the proteins, peptides, amino acids, sugars, lipids, phospholipids, inorganic salts and/or the isolation of trace components is performed on mobile, magnetic silica gel beads, wherein the beads having a bead diameter between 100 nm and 100 μm.

14. The trace components received by means of a purification process for proteins, peptides, amino acids, sugars, lipids, phospholipids and inorganic salts according to claim 13 wherein the surfaces of the beads include functional groups selected from the group consisting of, for example, OH, —COOH, NH₂, R—SO₂—OH, —NH₂; —RNH, —R₂N, —CH₃, —C₂H₅, —C₄H₉, —C₈H₁₇, —C₆H₅, —ZrO₂, TiO₂, C₆H₉NO₆, phenylhexyl, biphenyl, hydroxyapatite and boronic acid.

15. The trace components received by means of a purification process for proteins, peptides, amino acids, sugars, lipids, phospholipids and inorganic salts according to claim 13 wherein the binding of the proteins, peptides, amino acids, sugars, lipids, phospholipids, inorganic salts and the isolation of the trace components is achieved by a change in the dielectric constant by addition of at least one organic solution which has a dipole moment between 1.6 and 4.0 Debye at a volume fraction within a range between 1.5 and 4.
